(19) United States
(12) Patent Application Publication
(10) Pub. No.: US 2004/0064069 A1
(43) Pub. Date: Apr. 1, 2004

(54) Medical device with support member

(76) Inventors: Brian R. Reynolds, Ramsey, MN (US); Brice Shireman, Maple Grove, MN (US); Bonnie Laundry, Fridley, MN (US); Anthony C. Vrba, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 10/261,019

(22) Filed: Sep. 30, 2002

(51) Int. Cl. A61B 5/00; A61M 25/00

(52) U.S. Cl. 600/585

(57) ABSTRACT

A medical device having a support member disposed between an elongate shaft and a coil. The support member is adapted and configured to provide structural support to the coil so as to help maintain the position of the coil relative to the shaft.

### FIELD OF THE INVENTION

The invention pertains to medical devices and, more particularly, to medical devices, such as guidewires, catheters, or the like, having a support member disposed between an elongate shaft and a coil.

### BACKGROUND

A wide variety of medical devices have been developed for medical use, for example, intravascular use. Some of these devices include an elongated core or shaft having a coil disposed around a portion of the elongated core or shaft. In some such medical devices, at least a portion of the elongated core or shaft has an outer diameter that is less than the inner diameter of at least a portion of the coil, thereby leaving no or little support for a portion of the coil. Of the known medical devices that have a coil, each has certain advantages and disadvantages. There is an ongoing need to provide alternative guidewire structures and assemblies.

### SUMMARY

The invention provides design, material, and manufacturing method alternatives for medical devices having an elongate shaft or core, and a coil disposed about at least a portion of the elongate shaft or core. In at least some embodiments, the medical devices include a support member disposed between at least a portion of the elongate shaft or core and at least a portion of the coil that provides support to at least a portion of the coil so that it may, for example, maintain the position of at least a portion of the coil relative to at least a portion of the shaft.

### BRIEF DESCRIPTION OF THE DRAWINGS

FIG. 1 is a partial cross-sectional view of an example embodiment of a medical device including a support member disposed between an elongate shaft and a coil;

FIG. 2 is a partial cross-sectional view of another example embodiment of a medical device including a plurality of support members disposed between an elongate shaft and a coil;

FIG. 3 is a partial cross-sectional view of another example embodiment of a medical device including a support member having an alternative structure disposed between an elongate shaft and a coil;

FIG. 4 is a partial cross-sectional view of another example embodiment of a medical device including a plurality of support members disposed between an elongate shaft and a coil and also includes marker members disposed between the support members;

FIG. 5 is a partial cross-sectional view of another example embodiment of a medical device including a plurality of support members disposed between an elongate shaft and a coil and also includes marker members disposed between the support members;

FIG. 6 is a partial cross-sectional view of another example embodiment of a medical device including a support member having another alternative structure disposed between an elongate shaft and a coil;

FIG. 7 is a partial cross-sectional view of another example embodiment of a medical device including a support member having another alternative structure disposed between an elongate shaft and a coil;

FIG. 8 is a partial cross-sectional view of another example embodiment of a medical device including a support member having another alternative structure disposed between an elongate shaft and a coil; and

FIG. 9 is a transverse cross-sectional view of another example embodiment of a medical device including a support member having another alternative structure disposed between an elongate shaft and a coil.

### DETAILED DESCRIPTION

The following description should be read with reference to the drawings wherein like reference numerals indicate like elements throughout the several views. The detailed description and drawings illustrate some example embodiments of the claimed invention. As used herein, the term “about” applies to all numeric values, whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The invention relates to a medical device having a flexible distal tip. A number of different such medical devices, for example guidewires, catheters, and the like, are used in certain medical procedures and for treating many types of disease. For example, an intravascular device can be inserted into the vascular system of the patient and navigated through the vasculature to a desired target site. Using this method, virtually any target site in the patient's vascular system may be accessed, including, for example, the coronary, cerebral, and peripheral vasculature.

Some medical devices include a core portion surrounded at least partially by a coil. In some embodiments, the core portion includes at least a section having an outer surface or diameter that is smaller than the inner surface or diameter of at least a portion of the coil that surrounds it. For example, if the outer diameter of the core member is tapered, in some embodiments at least a portion of the outer diameter of the core will be smaller than the inner diameter of the coil, leaving a space or gap between the two. As such, there can be little or no support for portions of the coil. As a result, portions of the coil or individual turns of the coil can shift over one another and can become misaligned or otherwise undesirably shift positions. Misalignment of the coil could cause the guidewire to “catch” or “lock-up” or be generally difficult to pass through a lumen, for example the lumen of a catheter.

To help keep the coil in alignment as well as provide other useful features, some embodiments disclosed herein relate to a medical device having one or more support member disposed between an elongate core member and a coil that is disposed about the core member. In some embodiments, the one or more support member is disposed about the core, and has an outer surface having a size or diameter that is adapted and configured to come in contact with and provide support to at least a portion of the inner surface of the coil. In some embodiments, at least a portion of the outer surface of the one or more supports has a size or diameter that is substantially the same as the size or diameter of a portion of the inner surface of the coil. The one or more support member may come in a variety of different shapes and/or forms, and generally provides structural support to the coil so that the coil remains in the desired alignment.

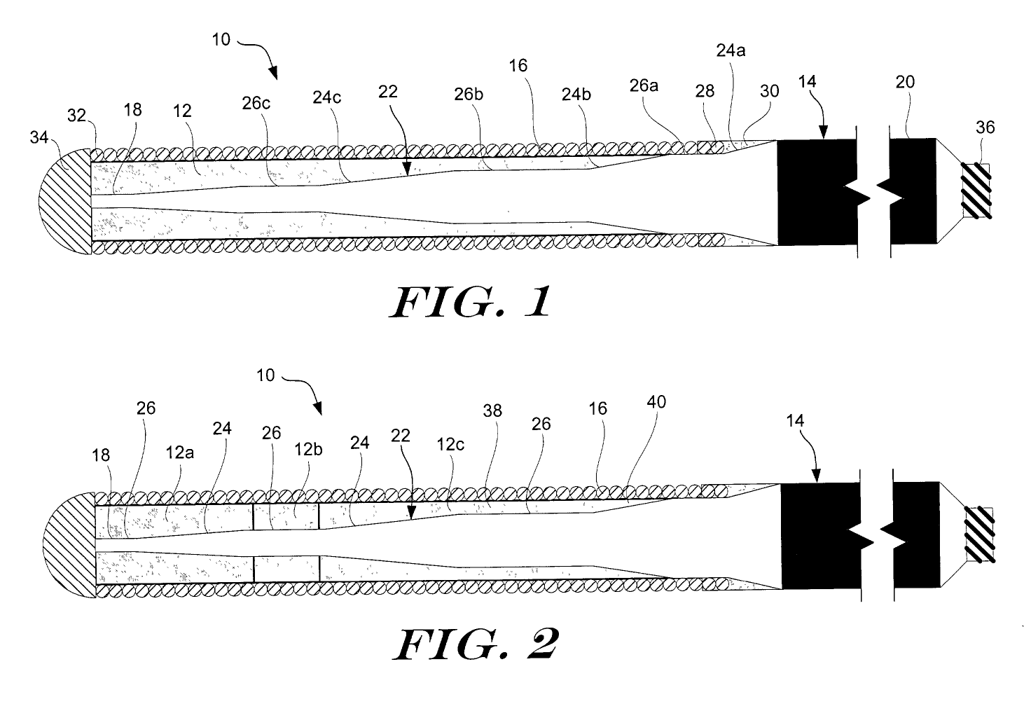

In the embodiments shown in FIGS. 1-6, the medical device is depicted as a guidewire. However, the invention is not intended to be limited to guidewires. It can be appreciated that the device could be any intravascular device or be any device designed to pass through an opening or body lumen. For example, the device may comprise a catheter (e.g., therapeutic, diagnostic, or guide catheter), endoscopic device, laproscopic device, an embolic protection device, and the like or any other such device.

Refer now to FIG. 1, which is a partial cross-sectional view of a medical device 10, which in this embodiment is a guidewire. The guidewire 10 includes an elongate core member or shaft 14, an outer coil 16, and at least one support member 12 disposed between a portion of the elongate shaft 14 and a portion of the coil 16. As will be discussed in more detail below, the at least one support member 12 may come in a variety of different shapes and/or forms, and generally provides structural support to coil 16 so that portions of the coil 16 remain in the desired alignment relative to one another or relative to the shaft 14.

The shaft 14 can be made of any suitable material including, for example, metals, metal alloys, polymers, metal-polymer composites, or the like, or combinations or mixtures thereof. Some examples of suitable metals and metal alloys include stainless steel, such as 304v stainless steel; nickel-titanium alloy, such as nitinol, nickel-chromium alloy, nickel-chromium-iron alloy, cobalt alloy, or the like; or other suitable material.

The shaft 14 may include a distal region 18 and a proximal region 20. The entire shaft 14 can be made of the same material, or in some embodiments, can include portions or sections made of different materials. In some embodiments, the material used to construct shaft 14 is chosen to impart varying flexibility and stiffness characteristics to different portions of shaft 14. For example, proximal region 20 and distal region 18 may be formed of different materials, for example materials having different moduli of elasticity, resulting in a difference in flexibility. In some embodiments, the material used to construct proximal region 20 can be relatively stiff for pushability and torqueability, and the material used to construct distal region 18 can be relatively flexible by comparison for better lateral trackability and steerability. For example, proximal region 20 can be formed of straightened 304v stainless steel wire or ribbon, and distal region 18 can be formed of a straightened super elastic or linear elastic alloy, for example a nickel-titanium alloy wire or ribbon.

In embodiments where different portions of shaft 14 are made of different material, the different portions can be connected using any suitable connecting techniques. For example, the different portions of the core wire can be connected using welding, soldering, brazing, adhesive, or the like, or combinations thereof. Additionally, some embodiments can include one or more mechanical connectors or connector assemblies to connect the different portions of the core wire that are made of different materials. The connector may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Some other examples of suitable techniques and structures that can be used to interconnect different shaft sections are disclosed in U.S. patent application Ser. No. 09/972,276, which is incorporated herein by reference.

The length of shaft 14, or the length of individual portions thereof, are typically dictated by the length and flexibility characteristics desired in the final guidewire. In some example embodiments, proximal portion 20 may have a length in the range of about 20 to about 300 centimeters and distal portion 18 may have a length in the range of about 3 to about 50 centimeters. It can be appreciated that alterations in the length of shaft 14 or portions thereof can be made without departing from the spirit of the invention. In addition, shaft 14 can have a solid cross-section as shown, but in some embodiments, can have a hollow cross-section. In yet other embodiments, shaft 14 can include a combination of areas having solid cross-sections and hollow cross sections. Moreover, the shaft, or portions thereof, can be made of rounded wire, flattened ribbon, or other such structures having various cross-sectional geometries. The cross sectional geometries along the length of the shaft can also be constant or can vary.

The shaft 14 may include one or more tapered regions 22, for example adjacent distal region 18. For example, in some embodiments the distal region 18 may be tapered and have an initial outside size or diameter that can be substantially the same as the outside diameter of the proximal region 20, which then tapers to a reduced size or diameter. For example, in some embodiments, the distal region 18 can have an initial outside diameter that is in the range of about 0.010 to about 0.020 inches, that tapers to a diameter in the range of about 0.001 to about 0.005 inches. Tapered region 22 may be linearly tapered, tapered in a curvilinear fashion, uniformly tapered, non-uniformly tapered, or tapered in a step-wise fashion. The angle of any such tapers can vary, depending upon the desired flexibility characteristics. The length of the taper may be selected to obtain a more (longer length) or less (shorter length) gradual transition in stiffness. Although FIG. 1 depicts tapered region 22 as being adjacent distal region 18, it can be appreciated that essentially any portion of shaft 14 may be tapered and the taper can be in either the proximal or the distal direction. As shown in FIG. 1, tapered region 22 may include one or more portions where the outside diameter is narrowing, for example, the tapered portions 24a/b/c, and portions where the outside diameter remains essentially constant, for example, constant diameter portions 26a/b/c. The number, arrangement, size, and length of the narrowing and constant diameter portions can be varied to achieve the desired characteristics, such as flexibility and torque transmission characteristics. The narrowing and constant diameter portions as shown in FIG. 1 are not intended to be limiting, and alterations of this arrangement can be made without departing from the spirit of the invention.

The tapered and constant diameter portions of tapered region 22 may be formed by any one of a number of different techniques, for example, by centerless grinding methods, stamping methods, and the like. The centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing shaft 14 during the grinding process. In some embodiments, shaft 14 is centerless ground using a Royal Master HI-AC centerless grinder.

The coil 16 can be disposed about at least a portion of shaft 14. In at least some embodiments, the coil 16 is disposed about the shaft 14 such that at least a portion of the coil 16 has an inner surface having a size or diameter that is greater the size or diameter of at least a portion of the outer surface of the elongated shaft 14. For example, coil 16 may be disposed about distal region 18 and can include a portion disposed about one or more of the tapered regions 22. As such, a space or gap is formed between at least a portion of the coil 16 and at least a portion of the shaft 14.

The coil 16 can be made of any or a variety of suitable materials, including, for example, metals, metal alloys, polymers, metal-polymer composites, and the like. Some examples of materials include stainless steel, nickel-chromium alloy, nickel-chromium-iron alloy, cobalt alloy, platinum, or other suitable materials, and the like. Some additional examples of suitable material include straightened super elastic or linear elastic alloy (e.g., nickel-titanium) wire, or alternatively, a polymer material, such as a high performance polymer. In some embodiments, coil 16 can be made of ,in full or in part, coated with, or doped with a radiopaque material.

Coil 16 may be formed of round wire or flat ribbon ranging in dimensions to achieve the desired characteristics, such as flexibility, and be wrapped in a generally helical fashion by conventional winding techniques. The pitch of adjacent turns of coil 16 may be tightly wrapped so that each turn touches the succeeding turn or the pitch may be set such that coil 16 is wrapped in an open fashion. Moreover, the pitch of the coil can be varied along the length device 10. In some embodiments, a coating, for example a lubricious (e.g., hydrophylic) or other type of coating may be applied over portions or all of coil 16. Some examples of such coatings include those discussed below with regard to coatings that can be used on the support member 12. Additionally, the thickness of the coil may be varied along the longitudinal axis of the device 10.

Coil 16 may include a proximal end 28 that is coupled to or otherwise attached to shaft 14. The coil 16 can be attached using suitable attachment mechanism, for example a solder joint 30 or other suitable attachment means such as adhesive, thermal bonding, mechanical bonding, and the like. A distal end 32 of coil 16 may be coupled to shaft 14, for example, by a distal solder ball tip 34 or other suitable connection. It is also of note that in embodiments where device 10 is a guidewire, device 10 may include some of the other structural features of guidewires. For example, device 10 may include proximal connector 36.

In order to incorporate other desirable properties into device 10, for example improve distal flexibility, coil 16 may taper inward toward shaft 14. For example, coil 16 may define the outside diameter of a portion of device 10, and the outside diameter may be greater near proximal end 28 of coil than at distal end 32 of coil 16.

The support member 12 is generally disposed about at least a portion of shaft 14, and includes at least a portion thereof that is disposed between the coil 16 and the shaft 14. It can be appreciated that the one of the functions of support member 12 is to provide structural support to coil 16 where a gap between shaft 14 and coil 16 may occur. For example, if coil 16 is disposed adjacent tapered region 22 of shaft 14, a gap or space is formed between the shaft 14 and the coil 16, and portions of the coil 16 or individual windings of coil 16 adjacent tapered region 22 can become displaced and, possibly, impair function of device 10 as alluded to above. Thus, in some embodiments, support member 12 can be disposed adjacent a region between the shaft 14 and coil 16 where the outside diameter of the shaft 14 is less than the inside diameter of the coil. The support member 12 can be disposed over a portion of the tapered region 22 to bridge shaft 14 and coil 16.

The support member 12 may be a variety of different shapes, forms, and/or sizes, dependent upon the shape, form, and/or size necessary to provide the desired structural support to coil 16 and include the desired characteristics. In some embodiments, the shape of support member 12 may be one that essentially fills at least a portion of the gap between shaft 14 and coil 16. For example, support member 12 may follow the contour of shaft 14 and taper proximally in a manner essentially opposite to the shape of tapered region 22. The shape of support member 12 as shown in FIG. 1, thus, provides structural support to coil 16 along at least some of the positions where a gap between coil 16 and shaft 14 might otherwise be present.

The support member 12 may also have a length or shape that is adapted and configured to span at least a portion of the length of tapered region 22 of shaft 14 and/or coil 16. For example, support member 12 may essentially span the length of a tapered region 22 and/or coil 16. This feature allows support member 12 to provide structural support to coil 16 along essentially the entire length of tapered region 22 where a gap would exist between shaft 14 and coil 12. However, the precise dimensions of support member 12 can be modified as desired, and can be dependent upon the particular configuration of shaft 14 and coil 16. For example, a first narrowing region 24a of tapered region 22 may decrease the outside diameter of shaft 14 so that proximal end 28 coil 16 may be attached to shaft 14, for example, adjacent first narrowing region 24a and/or at a first constant region 26a. This configuration allows coil 16 to be attached to shaft 14 essentially without adding to the outside diameter of device 10. Because support member 12 is configured to provide support to coil 16 between coil 16 and shaft 14, and because proximal end 26 of coil 16 may be coupled to shaft 14 adjacent first narrowing region 24a, and/or first constant region 26a, it may not be necessary for support member 16 to be disposed between coil 16 and shaft 14 at this particular portion of tapered region 22. It can be seen in FIG. 1 that at a second narrowing region 24b, a gap begins to form between coil 16 and shaft 14. Thus, according to this embodiment, the proximal end of support member 12 may begin essentially at this position (i.e., where the gap begins to form).

Other embodiments of device 10 may include coil 16 being coupled to the outside surface of shaft 14 adjacent a non-tapered portion. According to these embodiments, support member 12 may then be disposed essentially along the entire length of tapered region 22 and/or coil 16. It can be appreciated that similar configurations of support member 12, shaft 14, and coil 16 may occur at distal end 32 of coil, for example if the direction of the taper is altered.

In yet some other embodiments, support member 12 does not necessarily extend the entire length of the gap formed between the shaft 14 and the coil 16. For example, one or more support members 12 may extend only within a portion of the gap formed between the shaft 14 and coil 16. For example, in some embodiments, the one or more support members 12 may occupy in the range of about ¾ or more, ½ or more, or ¼ or more of the entire length of the gap formed between the coil 16 and shaft 14. As alluded to above, in some embodiments, more than one support member can be used.

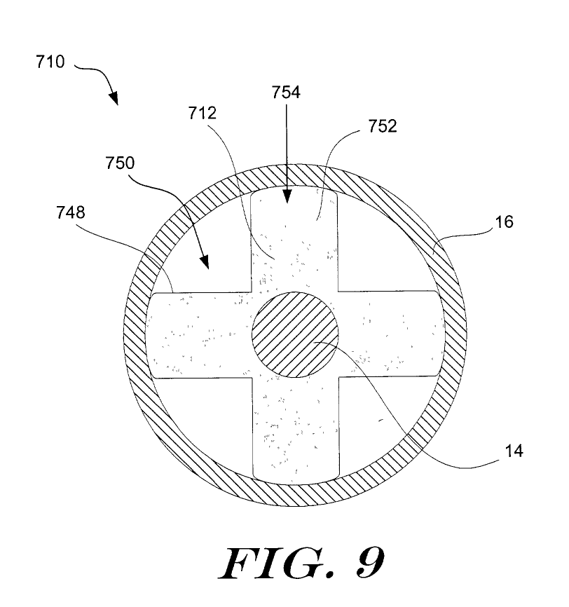

In some embodiments, support member 12 may comprise a sheath or tubular member that can be disposed over a portion of the shaft 14. Alternatively, support member 12 may comprise a coil or other suitable structure that can provide structural support to coil 16. The cross-sectional shape of the support member 12 can be any shape that provides the desired amount of support for the coil, and other desired characteristics, such as flexibility characteristics. In some embodiments, the support member 12 is generally circular in cross section. In some other embodiments, the cross-sectional shape of the support member could be generally oval, square, rectangular, triangular, or other such geometries. In some embodiments, the cross-sectional shape of the support member could include one or more protrusions that are adapted and configured to support a portion of the inner surface of the coil. For example, the embodiment shown in FIG. 9, as will be discussed in more detail below, the cross-sectional shape of the support member 712 includes four protrusions 752 that are adapted and configured to contact the inner surface of the coil 16. It can be appreciated that in other embodiments, the support member could include more or fewer such protrusions 752, as desired, to achieve the desired level of support and other characteristics, such as flexibility.

Support member 12 may be made with materials such as polymers, metals, metal alloys, metal-polymer composites, or other suitable materials. Some examples of suitable polymers may include PTFE, polyurethane, polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), silicones, polyethylene, Marlex high-density polyethylene, linear low density polyethylene (for example REXELL®), polyetheretherketone (PEEK), polyimide (PI), polyetherimide (PEI), or mixtures, combinations, or copolymers thereof. In some embodiments support member 12 can include a liquid crystal polymer (LCP) blended with other polymers to enhance torqueability. By employing selection of materials and processing techniques, thermoplastic, solvent soluble, and thermosetting variants of these and other materials can be employed to achieve the desired results.

In some embodiments, a coating, for example a lubricious, a hydrophilic, a protective, or other type of coating may be applied over portions or all of support member 12, or other portions of device 10. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Support member 12, or portions thereof, may also be doped with or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, plastic material loaded with a radiopaque filler, and the like.

Support member 12 may be disposed about or otherwise coupled to shaft 14 by any one of a number of different methods, depending somewhat upon the type of material used to construct the support member 12. For example, support member 12 may be coupled to shaft 14 by extrusion, casting, injection molding, adhesive bonding, mechanical bonding, thermal bonding, heat shrink techniques, and the like, or combinations thereof. In one particular example embodiment, support member 12 may comprise a PTFE heat-shrink tube that can be heat-shrunk over at least a portion of tapered region 22 of the shaft 14.

It can be appreciated that support member 12 can be a single layer or a plurality of layers. For example, support member 12 can be include one or more layers disposed or stacked on top of one another. The different layers may be made of the same material or different materials. In one example, one of the layers may be made of a generally less flexible polymer than the other(s). In another example, one or more of the layers may be doped with a radiopaque material.

Refer now to FIG. 2, which shows one example wherein a plurality of support members 12a/b/c may be disposed between shaft 14 and coil 16. Support members 12a/b/c can be made of the same material or of different materials. For example, support member 12c may be made of a metal or polymer, support member 12b may be made of a more flexible metal or polymer, and support member 12c may be made of an even more flexible metal or polymer. Moreover, one or more of support members 12a/b/c may be doped with or made of radiopaque materials.

The shapes of support members 12a/b/c may also be different. For example, support member 12b may be disposed along constant region 26 and, thus, be generally tubular and essentially without any tapered regions. In contrast, support members 12a/c may be disposed about both constant region 26 and narrowing region 24 of tapered region 22. For example, support members 12c may have a shape that includes both a non-tapered portion 38 and a tapered portion 40. It can be appreciated that the shapes illustrated for support members 12a/b/c may be used where appropriate in other example embodiments.

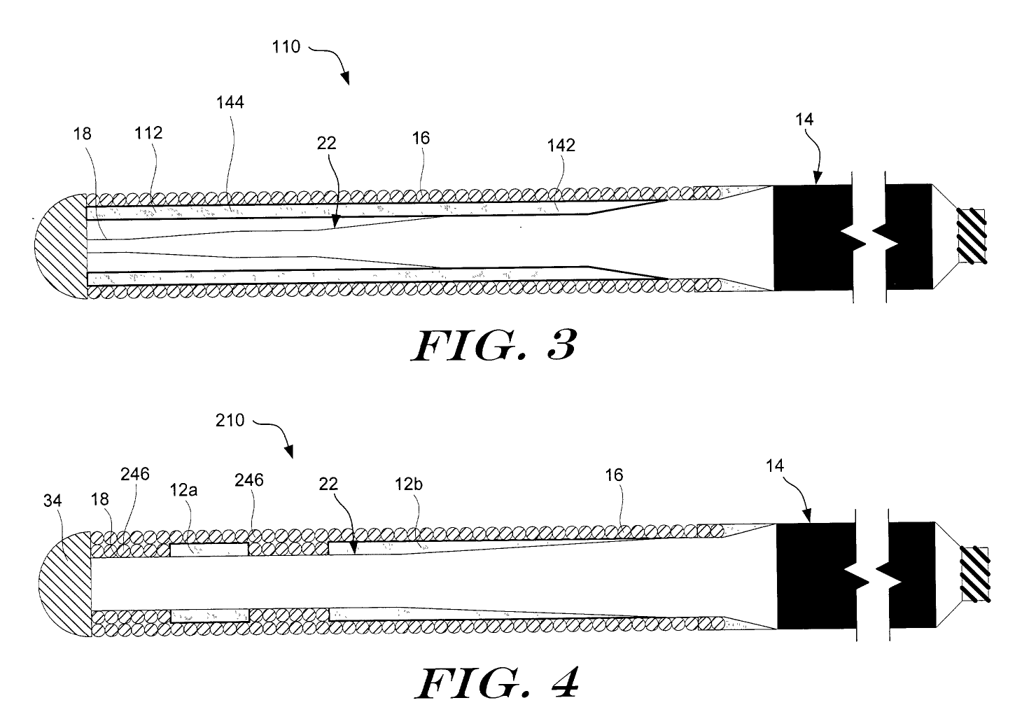

Refer now to FIG. 3, which depicts an example of a medical device 110 that is similar to device 10, but wherein the support member 112 is a generally tubular member disposed annularly around a portion of the shaft 14. The support member 112 includes a first portion 142 coupled to shaft 14 and a second portion 144 that is not coupled to shaft 14. In this example, support member 112 may be configured a distance away from shaft 14 at second portion 144 to form a space or gap between the support member 112 and the shaft 14. Such a configuration, or modifications thereof, may provide desirable features to medical device 110, for example, desirable flexibility characteristics.

FIG. 4 depicts another example embodiment of a medical device 210 that is similar to the device 10, except device 210 may include one or more marker members 246, for example radiopaque coils. According to the embodiment shown in FIG. 4, marker members 246 are disposed between shaft 14 and coil 16. In this example, a first support member 12a is disposed between the two marker member 246 and between a portion of the shaft 14 and a portion of the coil 16. Thus, first support member 12a provides structural support to coil 16 along shaft 14 between marker members 246. Also illustrated in this example is a second support member 12b that is located proximal to marker members 246 and disposed between a portion of the shaft 14 and a portion of the coil 16 to provide structural support to coil at remaining positions located proximal to marker members 246.

It can also be appreciated the position of support members 12a/b and/or marker member 246 could be altered. For example, one or more support members 12 may be disposed between distal tip 34 and marker member 246. Additionally, if a gap would exist between marker members 246 and coil 16, support member 12 may be disposed within the gap and provide support to coil. Similarly, if marker members 246 are coupled to coil 16 and, thus, a gap would exist between marker members 246 and shaft 14, then support member 12 may be disposed within this gap. Moreover, marker members 246 could also be embedded, disposed within, or otherwise coupled to support member 12. According to this embodiment, support member 12 can also provide structural support to coil 16.

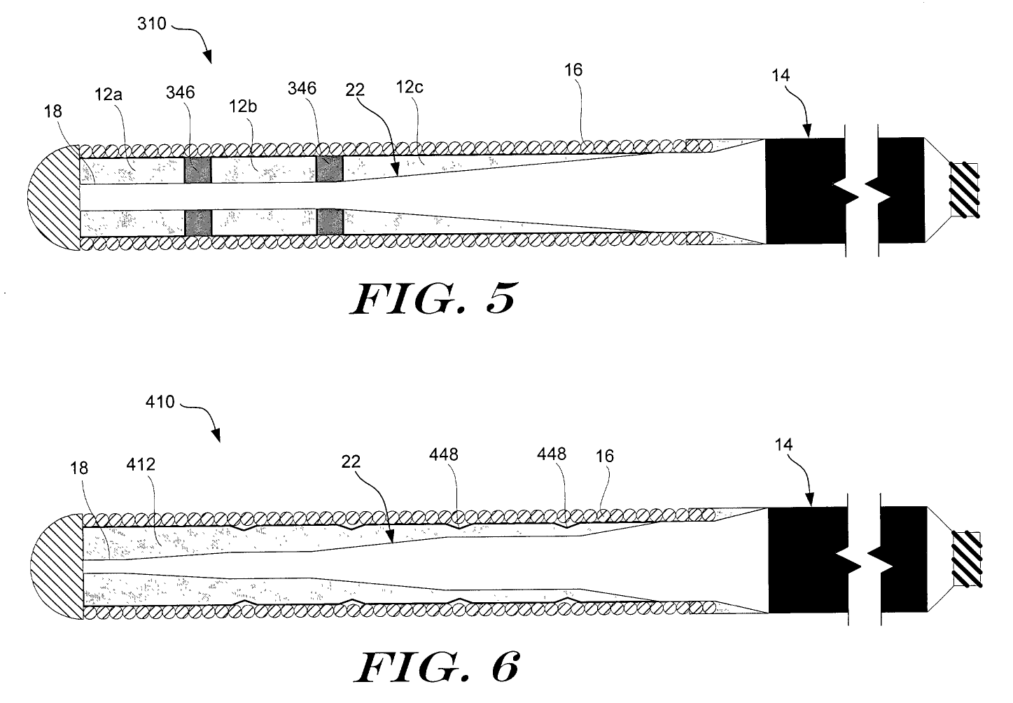

FIG. 5 is a partial cross-sectional view of another example embodiment of a medical device 310 that is similar to the previously described medical device except that marker members 346 comprise marker bands. Marker members 346 may be metallic bands that are similar or analogous to marker bands generally known in the art. In some examples, marker members 346 may be support members (like support members 12a/b/c) that are doped with radiopaque materials. In another example, marker members 346 may be a marker band coupled to or embedded within a support member 12.

FIG. 6 is a partial cross-sectional view of another example embodiment of a medical device 410 that is similar to previously described devices except that the exterior surface of support member 412 includes one or more inward deflections 448 formed in the outer surface of support member 412. These inward deflections 448 form channels or grooves in the outer surface of the support member 412. The deflections 448 can be disposed about the outer surface of the support member 412 to provide desired characteristics, for example, flexibility characteristics, to the support member and the medical device 410.

In some embodiments, inward deflections 448 can be separate grooves that are annularly disposed about the outer surface of the support member 412. In some embodiments, the inward deflections can be a single or multiple continuous grooves that are spirally disposed about the outer surface of support member 412. In some such embodiments, this may help to provide more even support to coil 16 and/or improve flexibility. In some other examples, inward deflections 448 may be formed or defined by twisting or spiraling support member 412 relative to shaft 14.

FIG. 7 is a partial cross-sectional view of another example embodiment of a medical device 510 that is similar to device 410 except that inward deflections 558 of support member 512 are widened or squared relative to deflections 448. Similar to what is described above, deflections 558 may improve desired characteristics of the medical device, for example, improve flexibility. Moreover, in some examples, support member 512 may be twisted or spiraled relative to shaft 14.

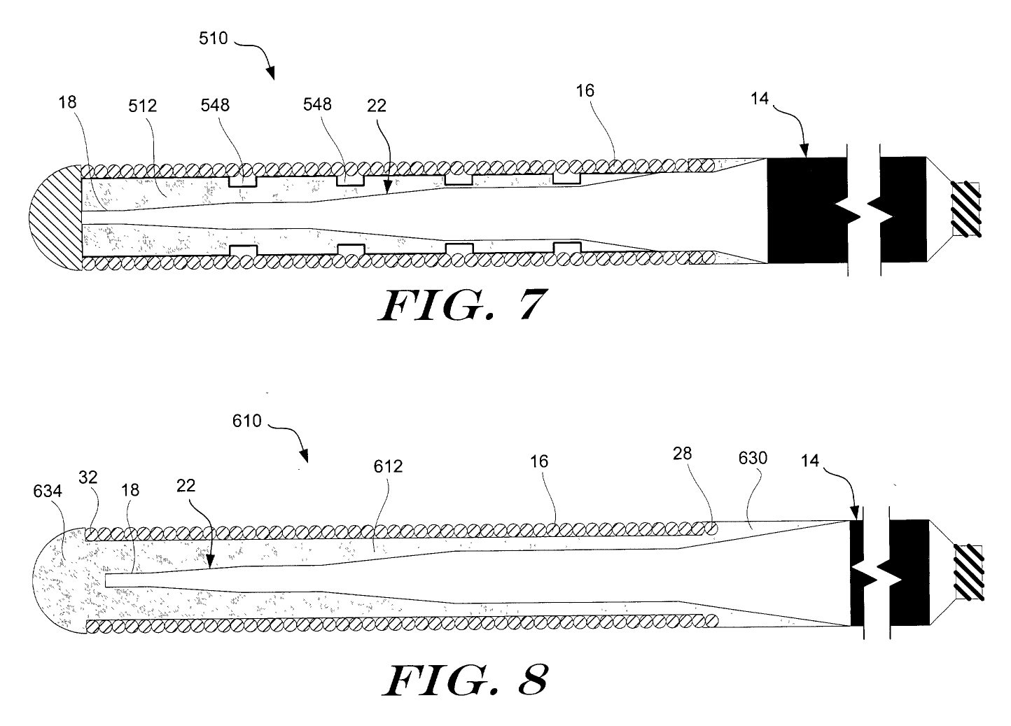

FIG. 8 is a partial cross-sectional view of another example embodiment of a medical device 610 that is similar to the devices described above, except that support member 612 extends distally beyond distal end 32 of coil 16. According to this embodiment, a portion of support member 612 may form a distal tip 634 of the medical device 610. This feature, for example, may allow support member 612 to both provide support to coil 16 and provide an atraumatic distal tip.

Additionally, support member 612 may extend proximally beyond proximal end 28 of coil 16. According to this embodiment, a proximal portion 630 of support member 612 may be coupled to the proximal end 28 of coil 16, and is disposed between the proximal end of the coil 16 and the shaft 14. In some embodiments, this may provide several desirable features to medical device 610, for example, improved flexibility.

FIG. 9 is a transverse cross-sectional view of another example medical device 710 that is similar to the devices described above, except that support member 712 has one or more inward deflections 748, and several protrusions 752 that extend radially outwardly from the longitudinal axis of the support member 712. The protrusions 752 are adapted and configured to contact the inner surface of the coil 16. Inward deflections 748 and the protrusions 752 may extend along the longitudinal axis of device 710 so as to define one or more longitudinal grooves 750 and one or more longitudinal ridges 754. In some embodiments, the inward deflections 748 and the protrusions 752 may extend generally parallel to each other along the longitudinal axis of device 710. In other embodiments, this is not the case. The embodiment shown includes four inward deflections 748 and four protrusions 752, but it can be appreciated that in other embodiments, the support member could include more or fewer such inward deflections 748 and protrusion 752, as desired, to achieve the desired level of support and other characteristics, such as flexibility. Additionally, it can be appreciated that the shape of the inward deflections 748 and protrusion 752 can be varied to achieve the desired level of support and other characteristics, such as flexibility.

Similar to what is described above, grooves 750 and ridges 754 may be configured to spiral or twist around the longitudinal axis of the support member 712. This may provide more even support to coil 16 or improve flexibility of device 710. Moreover, support member 712 may be twisted relative to shaft. This can also cause grooves 750 and ridges 754 to spiral or twist around the longitudinal axis of support member 712 and may incorporate some of the desired features described above.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A medical device, comprising:
an elongated shaft having an outer surface;
a coil disposed about at least a section of the shaft, the coil having an inner surface, wherein at least a portion of the inner surface of the coil is radially spaced from at least a portion of the outer surface of the shaft such that a space is defined between the portion of the inner surface of the coil the portion of the outer surface of the shaft;
a support member disposed within the space between the portion of the outer surface of the shaft and the portion of the inner surface of the coil.

2. The medical device of claim 1, wherein the portion of the coil is radially spaced from the portion of the shaft along a selected length, and the support member is disposed between the portion of the shaft and the portion of the coil for at least ¼ of the selected length.

3. The medical device of claim 1, wherein the portion of the coil is radially spaced from the portion of the shaft along a selected length, and the support member is disposed between the portion of the shaft and the portion of the coil for at least ½ of the selected length.

4. The medical device of claim 1, wherein the portion of the coil is radially spaced from the portion of the shaft along a selected length, and the support member is disposed between the portion of the shaft and the portion of the coil for at least ¾ of the selected length.

5. The medical device of claim 1, wherein the portion of the coil is radially spaced from the portion of the shaft along a selected length, and the support member is disposed between the portion of the shaft and the portion of the coil for the entire selected length.

6. The medical device of claim 1, wherein the portion of the elongated shaft includes at least a one tapered section that has a tapered outer diameter.

7. The medical device of claim 6, wherein the elongated shaft has a distal end and a proximal end, and the outer diameter of the at least a one tapered section reduces in outer diameter in a distal direction.

8. The medical device of claim 1, wherein the support member is adapted and configured to maintain the position of the coil relative to the shaft.

9. The medical device of claim 1, wherein the support member has an outer surface that is adapted and configured to fit within and be in contact with the inner surface of the coil.

10. The medical device of claim 1, wherein the support member has an inner surface that is adapted and configured to fit over and be in contact with the outer surface of the shaft.

11. A medical device, comprising:
an elongated shaft having a portion with an outer diameter;
a coil disposed about at least the portion of the shaft, the coil having at least a portion that has an inner diameter that is greater than the outer diameter of the portion of the shaft such that the portion of the coil is radially spaced from the portion of the shaft;
a support member disposed between the portion of the shaft and the portion of the coil.

12. The medical device of claim 11, wherein the portion of the coil is radially spaced from the portion of the shaft for a selected length, and the support member is disposed within the space for at least half of the selected length.

13. A medical device, comprising:
an elongate shaft having a tapered distal region;
a coil disposed about at least a portion of the tapered region; and
a support member disposed between the shaft and the coil along at least a portion of the tapered region.

14. The medical device in claim 13, wherein the support member is adapted and configured to maintain the position of the coil relative to the shaft.

15. The medical device in claim 13, further comprising a marker member disposed between the shaft and the coil.

16. The medical device in claim 13, wherein the support member includes a tapered region.

17. The medical device in claim 16, wherein at least a portion of the tapered region of the support member is adapted and configured to mate with at least a portion of the tapered distal region of the shaft.

18. The medical device in claim 13, wherein the support member includes a non-tapered region.

19. The medical device in claim 13, wherein the support member includes both a tapered region and a non-tapered region.

20. The medical device in claim 13, wherein the support member includes a surface that is in continuous contact with the shaft.

21. The medical device in claim 13, wherein the support member includes a surface that is in continuous contact with the coil.

22. The medical device in claim 13, wherein the support member includes a first surface that is in continuous contact with the shaft and a second surface that is in continuous contact with the coil.

23. The medical device in claim 13, wherein the support member includes an exterior surface adjacent the coil and wherein the exterior surface has a first portion in contact with the coil and a second portion not in contact with the coil.

24. The medical device in claim 13, wherein the coil has a length and wherein the support member substantially spans the length of the coil.

25. The medical device in claim 13, wherein the support member comprises one or more polymers.

26. The medical device in claim 25, wherein the polymer includes polytetrafluoroethylene.

27. The medical device in claim 13, wherein the support member comprises a metal or metal alloy.

28. The medical device in claim 13, further comprising a second support member.

29. A guidewire, comprising:
a core wire having a tapered distal region;
a coil disposed around at least a portion of the distal region, the coil having a proximal end, a distal end, and a length; and
a support member disposed between the core wire and the coil, the support member substantially spanning the length of the coil.

30. The medical device in claim 29, wherein the support member is adapted and configured to maintain the position of the coil relative to the core wire.

31. The medical device in claim 29, wherein the support-member includes a tapered region.

32. The medical device in claim 31, wherein at least a portion of the tapered region of the support member is adapted and configured to mate with at least a portion of the tapered distal region of the core wire.

33. The medical device in claim 29, wherein the support member includes a non-tapered region.

34. The medical device in claim 29, wherein the support member includes both a tapered region and a non-tapered region.

35. The medical device in claim 29, wherein the support member includes a surface that is in continuous contact with the core wire.

36. The medical device in claim 29, wherein the support member includes a surface that is in continuous contact with the coil.

37. The medical device in claim 29, wherein the support member includes a first surface that is in continuous contact with the core wire and a second surface in continuous contact with the coil.

38. The medical device in claim 29, wherein the support member includes an exterior surface adjacent the coil and wherein the exterior surface has a first portion in contact with the coil and a second portion not in contact with the coil.

39. The medical device in claim 29, wherein the support member comprises one or more polymers.

40. The medical device in claim 39, wherein the polymer includes polytetrafluoroethylene.

41. The medical device in claim 29, further comprising a second support member.

42. The medical device in claim 29, wherein a portion of the support member extends distally beyond the distal end of the coil.

43. The medical device in claim 29, wherein a portion of the support member extends proximally beyond the proximal end of the coil.

44. A medical device, comprising:
an elongate shaft having a tapered distal region;
a coil disposed about at least a portion of the distal region;
wherein at least a portion of the coil is spaced from the shaft; and
a support member disposed between the shaft and the coil.

45. A guidewire, comprising:
an elongate core wire having a tapered distal region;
a support member disposed over the tapered distal region; and
a coil disposed over at least a portion of the support member.

46. A guidewire, comprising:
an elongate core wire having a tapered region;
a coil disposed about at least a portion of the tapered region; and
means for maintaining the position of the coil relative to the core wire.

47. A method of manufacturing a guidewire, comprising the steps of:
providing an elongate core member having a proximal region and a tapered distal region;
disposing a support member over the tapered distal region; and
disposing a coil over the support member.

48. The method in claim 47, wherein the tapered distal region has a tapered outside diameter and wherein the step of disposing a support member over the tapered distal region includes evening at least a portion of the outside diameter of the tapered distal region.
